(19) United States
(12) Patent Application Publication
(10) Pub. No.: US 2008/0287668 A1
(43) Pub. Date: Nov. 20, 2008

(54) Nanostructures and methods of making

(76) Inventor: Tihamer Thomas Toth-Fejel, Ann Arbor, MI (US)

Correspondence Address: STEPTOE & JOHNSON LLP, 1330 CONNECTICUT AVENUE, N.W., WASHINGTON, DC, 20036, US

(21) Appl. No.: 11/798,396

(22) Filed: May 14, 2007

(51) Int. Cl. C07H 21/04

(52) U.S. Cl. 536/25.3; 536/22.1; 977/743

(57) ABSTRACT

A nanostructure can include a first single-stranded nucleic acid, a plurality of complementary single-stranded helper nucleic acids hybridized with the first single-stranded nucleic acid, and at least one nanocube linked to a helper nucleic acid by a tether. The nanostructure can include at least two nanocubes each linked to a helper nucleic acid by a tether, and bonded to each other.

### TECHNICAL FIELD

This invention relates to nanostructures and methods of making the nanostructures.

### BACKGROUND

Many nanostructures (e.g., semiconductor microprocessors) are assembled by a “top-down” process, where materials and components are prepared and assembled using large tools like scanning probe microscopes, microlithography machines, and nanoimprint. “Bottom-up” methods instead rely on molecular recognition and self-assembly, so that multiple molecular components act as their own tools. Often, they only need to can be combined in a single vessel and allowed to interact, but for more complex products, a step-wise process is necessary for sufficient control. The interactions (which are chosen to give a desired outcome) cause the desired structure to self-assemble from the components. Such bottom-up methods can be used with nucleotide structures (e.g., DNA or RNA) which are well known to spontaneously hybridize into complex structures based on sequence-specific hybridization. Some natural examples of complex nucleic structures include DNA and RNA hairpins, transfer RNA, and ribosomal RNAs.

### SUMMARY

The ability to make oligonucleotides of desired sequence, coupled with the knowledge of the sequence of large single stranded polynucleotides (e.g., certain viral genomes) and careful design can be harnessed to make complex, two- or three-dimensional structures in a process referred to as DNA origami. DNA origami structures can be further elaborated with modified bases that link to functional components, e.g., nanocubes. Positioning of the nanocubes within the three-dimensional DNA origami framework can be refined by tethering the nanocubes to the modified bases with a tether, such as an exact-length tether.

In one aspect, a nanostructure includes a first single-stranded nucleic acid, a plurality of complementary single-stranded helper nucleic acids hybridized with the first single-stranded nucleic acid, and at least one nanocube linked to at least one helper nucleic acid by a tether.

The first single-stranded nucleic acid (or scaffold nucleic acid) can be of biological origin (e.g., a viral genome, a single strand of a bacterial plasmid, a ribosomal RNA, or other biologically-derived nucleic acid) or of synthetic origin (e.g., formed by PCR, chemical synthesis, or formed by another synthetic method). In some embodiments, the scaffold nucleic acid is long (e.g., more than 500 bases long, more than 1,000 bases long, more than 2,000 bases long, more than 5,000 bases long, more than 10,000 or longer). The helper strands can be of biological or synthetic origin; in some embodiments the helper strands will be substantially prepared by synthetic methods. The helper strands are typically much shorter (e.g., less than 20 bases long, less than 50 bases long, less than 100 bases long, or less than 500 bases long; in some cases, helper strands can be as short as 16 bases long), and can be of synthetic origin so that the sequence can be precisely chosen so that one section of the helper strand hybridizes selectively with one part of the scaffold, while another section of the same helper strand hybridizes selectively with another part of the scaffold (thereby folding the scaffold in the desired manner).

The tether can be an exact-length tether. The tether includes a moiety of Formula (I):

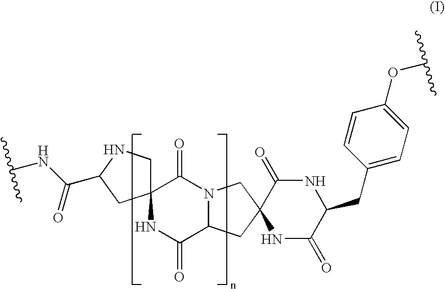

wherein n is in the range 0-50. In some embodiments, n can be in the range 0-40; in the range 0-30; in the range 0-20; or in the range 0-10.

The plurality of helper nucleic acids can include at least one helper nucleic acid substantially free of hairpins. The plurality of helper nucleic acids can include at least one helper nucleic acid including at least one hairpin. A helper nucleic acid can have at least two regions that each hybridize to different locations of the first single stranded nucleic acid. Thus, the helper nucleic acids can, by simultaneously hybridizing to two or more distant regions of the first single stranded nucleic acid, fold the first single stranded nucleic acid.

The nanostructure can include at least two nanocubes in proximity with one another. The nanostructure includes a nanocube in proximity with at least two other nanocubes. Two nanocubes can be “in proximity” to one another when they are, for example, within 10 nm, within 50 nm, within 100 nm or within 500 nm of one another, as measured through space between the closest atoms of the nanocube pair. Two nanocubes can be “in proximity” to one another if they are in electrical communication with one another, e.g., via through-space or through-bond electron transfers (or a combination thereof). Two nanocubes can be “in proximity” to one another if they are in electrical communication with one another, e.g., where the electrical state of one nanocube (e.g., net charge, potential, or valence state) influences one or more electrical properties (e.g., potential, conductivity, resistance) of the other nanocube.

The nanostructure can include at least two nanocubes bonded to one another. The nanostructure can include a nanocube bonded to at least two other nanocubes. Two nanocubes can be “bonded to” to one another when they are, for example, linked via a ligand, sharing electrons in a covalent bond, attracted by an ionic or hydrogen bond, or some a combination thereof.

At least one helper strand can include a first sequence hybridized to a first sequence of the first single-stranded nucleic acid, and a second sequence hybridized to a second sequence of the first single-stranded nucleic acid. The first and second sequences of the first single-stranded nucleic acid are separated by at least 10 nucleotides, at least 100 nucleotides, at least 250 nucleotides, or at least 500 nucleotides. In some instances, the first and second sequences of the first single-stranded nucleic acid can be adjacent (e.g., at the edges of the nanostructure), or, in other instances can be separated by at much as the first single-stranded nucleic acid (however, if the first single-stranded nucleic acid is a circular nucleic acid, the maximum distance is half the total number of nucleotides in the first single-stranded nucleic acid).

In another aspect, a nanostructure includes a first single-stranded nucleic acid, a first plurality of complementary single-stranded helper nucleic acids hybridized with the first single-stranded nucleic acid, wherein the helper strands of the first plurality each include a first sequence hybridized to a first sequence of the first single-stranded nucleic acid, and a second sequence hybridized to a second sequence of the first single-stranded nucleic acid. The first and second sequences of the first single-stranded nucleic acid are separated by at least 10 nucleotides. The nanostructure includes and a second plurality of complementary single-stranded helper nucleic acids hybridized with the first single-stranded nucleic acid. The helper strands of the second plurality each include a first sequence hybridized to a first sequence of the first single-stranded nucleic acid, a second sequence hybridized to a second sequence of the first single-stranded nucleic acid, and a hairpin intermediate the first and second sequences of the helper strand, and a plurality of nanocubes linked to helper nucleic acids by tethers.

The first and second sequences of the first single-stranded nucleic acid can be distinct sequences of nucleotides. By choosing distinct first and second sequences of the first single-stranded nucleic acid, it can be ensured that each helper strand will hybridize only with the desired sequences of the first single-stranded nucleic acid (and thus ensure that the desired fold is formed).

In another aspect, a method of making a nanostructure includes selecting a first single-stranded nucleic acid having a known sequence, determining a desired conformation for the first single-stranded nucleic acid, selecting, based on the desired conformation, a first plurality of helper sequences configured to hybridize to selected sequences of the first single-stranded nucleic acid, thereby folding the first single-stranded nucleic acid into the desired conformation, selecting a second plurality of helper sequences configured to hybridize to selected sequences of the first single-stranded nucleic acid, the second plurality of helper sequences each including at least one site capable of linking to a nanocube via a tether, and wherein the second plurality of helper sequences is selected to position linked nanocubes at desired locations in the nanostructure.

The method can further include selecting a tether for each site capable of linking to a nanocube to position linked nanocubes at desired locations in the nanostructure. Selecting the tether can include selecting a desired tether length.

The method can further include forming the first plurality of helper strands, the second plurality of helper strands, and linking at least one nanocube to at least one of the second plurality of helper strands.

The method can further include combining the first single-stranded nucleic acid, the first plurality of helper strands, and the second plurality of helper strands in a vessel, and allowing complementary sequences to hybridize, thereby forming the nanostructure.

The nanostructure formed by the method can include at least two nanocubes in proximity with one another. The method can include causing the at least two nanocubes in proximity with one another to become bonded to one another. Causing the at least two nanocubes in proximity with one another to become bonded to one another can include exposing the nanostructure to a trigger.

In another aspect, a method of making a nanostructure includes combining in a vessel: a first single-stranded nucleic acid having a known sequence, a first plurality of complementary single-stranded helper nucleic acids capable of hybridizing with the first single-stranded nucleic acid. The helper strands of the first plurality each include a first sequence capable of hybridizing to a first sequence of the first single-stranded nucleic acid, and a second sequence capable of hybridizing to a second sequence of the first single-stranded nucleic acid. The first and second sequences of the first single-stranded nucleic acid are separated by at least 10 nucleotides, and a second plurality of complementary single-stranded helper nucleic acids capable of hybridizing with the first single-stranded nucleic acid. The helper strands of the second plurality each include a first sequence capable of hybridizing to a first sequence of the first single-stranded nucleic acid, a second sequence capable of hybridizing to a second sequence of the first single-stranded nucleic acid, and a nanocube linked to the helper strand by a tether, and allowing the complementary sequences to hybridize, thereby forming the nanostructure.

Other features, objects and advantages will be apparent from the drawings, the description and the claims.

### BRIEF DESCRIPTION OF THE DRAWINGS

FIG. 1 is a schematic depiction of a portion of a nanostructure including three layers.

FIG. 2 schematically depicts triggers for bonding nanocubes.

### DETAILED DESCRIPTION

Silsesquioxane nanocubes (also called polyhedral oligomeric silsesquioxane (trademarked “POSS” by Hybrid Plastics) or simply “nanocubes”) are functionalized multi-generation silsesquioxanes, between 1 nm and 100 nm, or between 2 nm and 50 nm in size. See, for example, R. M. Laine, Nano-building blocks based on the [OSiO_{1.5}]₈ Silsesquioxanes, Journal of Materials Chemistry, 15, 3725-44 (2005), and U.S. Pat. No. 5,484,867, each of which is incorporated by reference in its entirety.

Nanocubes can be linked to DNA by specialized modifications to DNA, such as, for example, modifications to the bases, backbone sugars, and/or backbone phosphate linkages. In particular, a variety of modified bases have been prepared and described suitable for attaching further modifications to a nucleotide, oligonucleotide, or polynucleotide. Accordingly, a nucleotide, oligonucleotide, or polynucleotide can be prepared that has reactive functional groups available for attaching to other chemical moieties. Reactive functional groups include, but are not limited to, amines, thiols, alcohols, and carboxylic acids. A nucleotide, oligonucleotide, or polynucleotide can also be prepared that has an affinity group attached. An affinity group is one that binds to an affinity partner, usually non-covalently. Some well-known examples of affinity group/affinity partner pairs include biotin/streptavidin and FK506/FK506 binding protein.

“DNA origami” refers to the technique of using multiple oligonucleotides (helper strands) to fold a longer polynucleotide at desired locations in the scaffold, which is the longer sequence (see, e.g., Rothemund, P. W. K, “Folding DNA to create nanoscale shapes and patterns,” Nature, 2006, 440, 297-302, which is incorporated by reference in its entirety). For example, a circular, single stranded DNA (e.g., a viral genome) can be folded into a variety of shapes by selection of short, single-stranded “helper strands” that hybridize to the single stranded DNA at predetermined locations. In particular the helper strand can have at least two regions that each hybridize to different locations of the single stranded DNA. Thus, the helper strands can, by simultaneously hybridizing to two or more distant regions of the single stranded DNA, fold the single stranded DNA. DNA origami can therefore be used to controllably position different nucleotides of a large DNA complex relative to one another in two- or three-dimensional space. It is note that “DNA origami” can also be used with other nucleic acids (e.g., RNA) and nucleic acid analogs (e.g., peptide nucleic acids, PNA). The salient feature is the ability to have two strands hybridize in a sequence-specific manner.

DNA origami also includes the use of “pixilated” DNA origami. In pixilated DNA origami, some helper strands include a self-complementary hairpin “bump”. The helper strand hairpin is a nucleotide subsequence selected to hybridize with itself rather than with the single stranded DNA.

Nanocubes can be linked to DNA by a tether. The tether can be, for example, a polypeptide. In some embodiments, a polypeptide tether can be an exact-length tether (see, for example, Schafineister, C., et al. J. Am. Chem. Soc. 2003, 125, 4702-4703). An exact-length tether is a substantially inflexible chemical linker. Inflexibility helps ensure that the tether length does not vary due to, e.g., rotation of chemical bonds. Inflexibility can be provided by, for example, rings (such as three-, four-, five, six-, or seven-membered rings) or unsaturation (e.g., double or triple bonds, which may be conjugated), or a combination of rings and unsaturation (e.g., unsaturated rings such as a cyclopentenyl or cyclohexenyl ring, or aryl or heteroaryl rings).

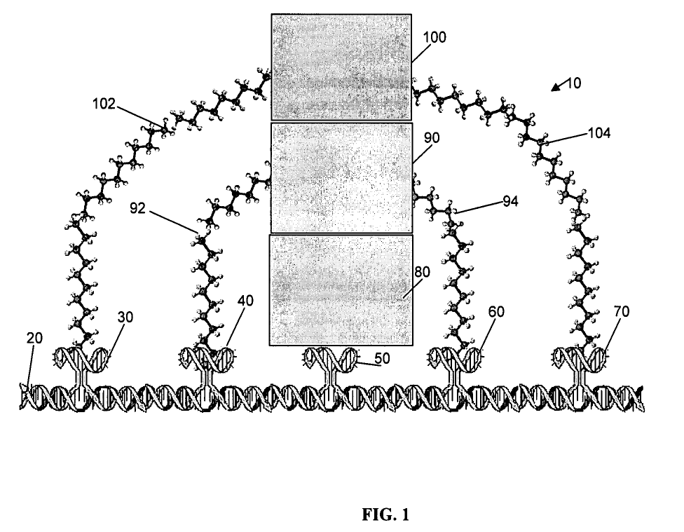

FIG. 1 is a schematic drawing illustrating a nanostructure 10. Structure 10 includes a first single stranded DNA 20, also referred to as a scaffold DNA 20, hybridized to helper strands 30, 40, 50, 60, and 70. In FIG. 1, the helper strands are shown with hairpins; in various embodiments, helper strands can all have hairpins, none have hairpins, or some have hairpins. A “hairpin” refers to the structure formed when a portion of a single nucleic acid strand has self-complementarity. Generally, the sequences flanking the hairpin will be complementary to a second nucleic acid strand, such that the hairpin takes the form of “bulge” in an otherwise ordinary double-stranded nucleic acid. In particular, in some embodiments, some helper strands have at least one hairpin, while other helper strands will not have any hairpins.

Helper strand 50 is linked to nanocube 80. Nanocube 80 is also positioned proximal to nanocube 90. Nanocube 90 is linked to helper strands 40 and 60 by tethers 92 and 94, respectively. Nanocube 90 is positioned proximal to nanocubes 80 and 100. Nanocube 100 (positioned proximal to nanocube 90) is linked to helper strands 30 and 70 by tethers 102 and 104, respectively.

Linkers 92, 94 and 102, 104 can be, for example, exact-length tethers derived from compounds such as those shown in Scheme 1. See, for example, Schafmeister, C., J. Am. Chem. Soc. 2003, 125, 4702-4703. Scheme 1 shows oligomers of lengths 1, 2, 3 and 4 units; longer oligomers are possible. When longer oligomers are used, additional helper strands may be required to position them.

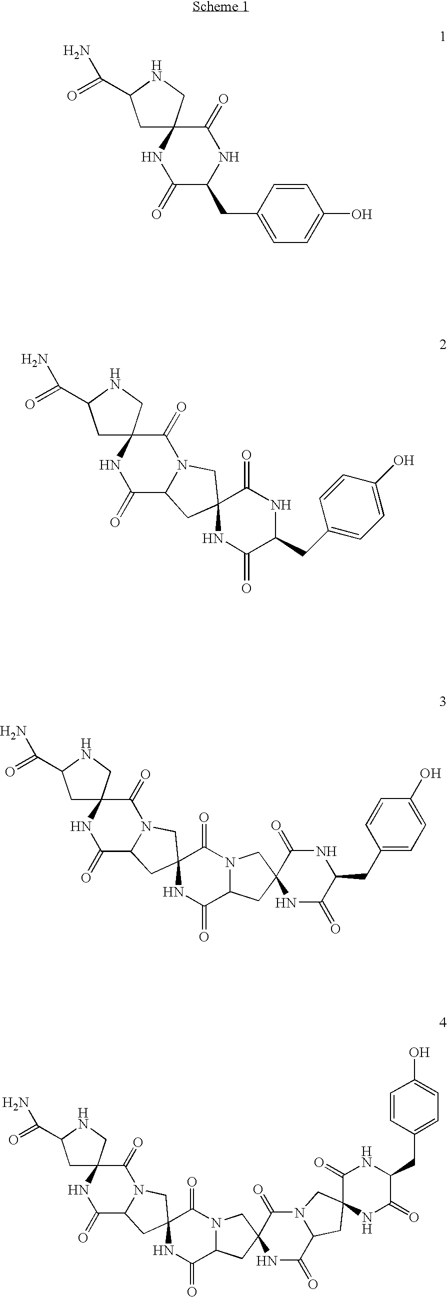

In other words, linkers 92, 94 and 102, 104 can include a moiety of formula (I):

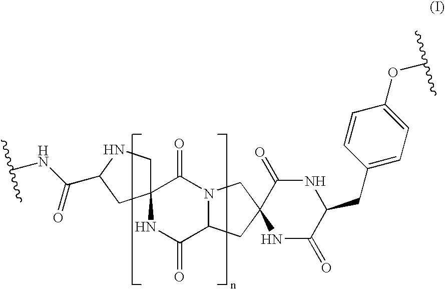

where n is in the range 0-50. In some embodiments, n can be in the range 0-40; in the range 0-30; in the range 0-20; or in the range 0-10. The tether can be covalently bonded to a helper strand. For example, the helper strand can include a modified base capable of forming covalent bonds (e.g., having a reactive amine or thiol) with a compound of the type shown in Scheme 1.

Nanocubes 80, 90, 100 can be chemically similar or chemically dissimilar. For example, nanocubes 80, 90, 100 can be selected for desirable properties, e.g., electrical properties. In one embodiment, nanocubes 80, 90, and 100 are selected such that nanocube 80 behaves as a semiconductor (e.g., an n-type or a p-type semiconductor), nanocube 90 behaves as an insulator, and nanocube 100 behaves as a conductor.

Thus, as illustrated schematically in FIG. 1, a variety of nanocubes can be controllably positioned in proximity to one another, in multiple layers of a two- or three-dimensional nanostructure. Two nanocubes can be “in proximity” to one another when they are, for example, within 10 nm, within 50 nm, within 100 nm or within 500 nm of one another, as measured through space between the closest atoms of the nanocube pair. Two nanocubes can be “in proximity” to one another if they are in electrical communication with one another, e.g., via through-space or through-bond electron transfers (or a combination thereof). Two nanocubes can be “in proximity” to one another if they are in electrical communication with one another, e.g., where the electrical state of one nanocube (e.g., net charge, potential, or valence state) influences one or more electrical properties (e.g., potential, conductivity, resistance) of the other nanocube.

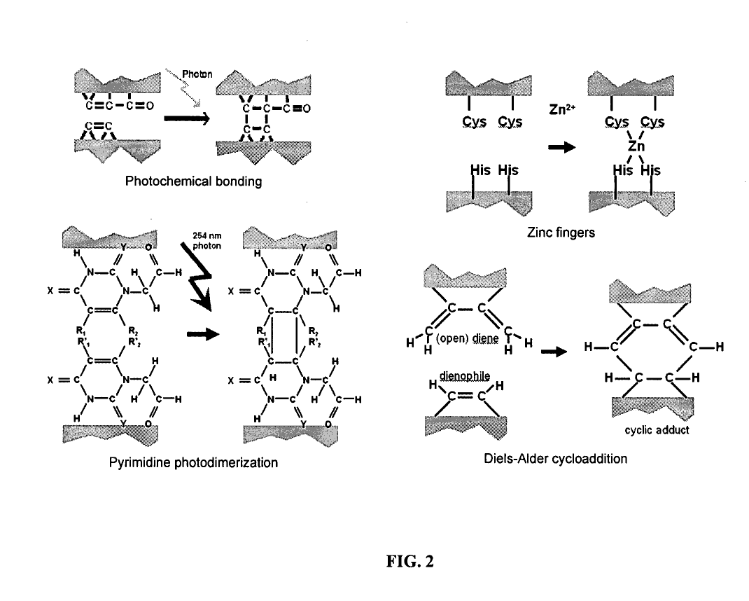

When two nanocubes are in sufficiently close proximity, a signal can trigger two nanocubes to bind to each other. Depending on the connection chemistry, this trigger can be photon of a particular wavelength, an electrically charged ion, a change of pH, or some other trigger. Four example connection chemistries are pictured in FIG. 2. They include photochemical bonding (upper left) (see Chris Phoenix and Tihamer Toth-Fejel, “Large-Product General-Purpose Design and Manufacturing Using Nanoscale Modules: Final Report”, CP-04-01, NASA Institute for Advanced Concepts, May 2005, http://www.niac.usra.edu/files/studies/final_report/1030Phoenix.pdf, which is incorporated by reference in its entirety); zinc fingers, which are protein motifs that selectively bind Zn²⁺ ions, (upper right) (see ibid); pyrimidine photodimerization (lower left) (see Brian Lohse, P. S. Ramanujam, Soren Hvilstead, and Rolf H. Berg. Photodimerization in pyrimidine-substituted dipeptides, Journal of Peptide Science. 11: 499-505 (2005) http://www.polymers.dk/publikation/pdf/101%20J%20Peptide%20Sci%2005.pdf, which is incorporated by reference in its entirety) and Diels-Alder cycloaddition (see Markus Krummenacker, “Steps Towards Molecular Manufacturing”, Chemical Design Automation News, 9, (1994) p. 1 and 29-39, http://www.n-a-n-o.com/nano/cda-news/cda-news.html, which is incorporated by reference in its entirety).

Other embodiments are within the scope of the following claims.

What is claimed is:

1. A nanostructure comprising:
a first single-stranded nucleic acid;
a plurality of complementary single-stranded helper nucleic acids hybridized with the first single-stranded nucleic acid; and
at least one nanocube linked to a helper nucleic acid by a tether.

2. The nanostructure of claim 1, wherein the tether is an exact-length tether.

3. The nanostructure of claim 1, wherein the tether includes a moiety of Formula (I): 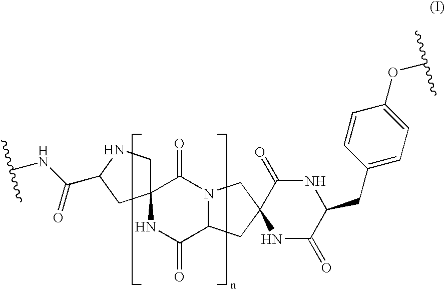
wherein n is in the range 0-50.

4. The nanostructure of claim 1, wherein the plurality of helper nucleic acids includes at least one helper nucleic acid substantially free of hairpins.

5. The nanostructure of claim 1, wherein the plurality of helper nucleic acids includes at least one helper nucleic acid including at least one hairpin.

6. The nanostructure of claim 1, wherein the nanostructure includes at least two nanocubes in proximity with one another.

7. The nanostructure of claim 6, wherein the nanostructure includes a nanocube in proximity with at least two other nanocubes.

8. The nanostructure of claim 1, wherein the nanostructure includes at least two nanocubes bonded to each other.

9. The nanostructure of claim 8, wherein the nanostructure includes a nanocube bonded to at least two other nanocubes.

10. The nanostructure of claim 1, wherein at least one helper strand includes:
a first sequence hybridized to a first sequence of the first single-stranded nucleic acid; and
a second sequence hybridized to a second sequence of the first single-stranded nucleic acid;
wherein the first and second sequences of the first single-stranded nucleic acid are separated by at least 10 nucleotides.

11. The nanostructure of claim 10, wherein the first and second sequences of the first single-stranded nucleic acid are separated by at least 100 nucleotides.

12. The nanostructure of claim 10, wherein the first and second sequences of the first single-stranded nucleic acid are separated by at least 250 nucleotides.

13. The nanostructure of claim 10, wherein the first and second sequences of the first single-stranded nucleic acid are separated by at least 500 nucleotides.

14. A nanostructure comprising:
a first single-stranded nucleic acid;
a first plurality of complementary single-stranded helper nucleic acids hybridized with the first single-stranded nucleic acid, wherein the helper strands of the first plurality each include:
a first sequence hybridized to a first sequence of the first single-stranded nucleic acid; and a second sequence hybridized to a second sequence of the first single-stranded nucleic acid; wherein the first and second sequences of the first single-stranded nucleic acid are separated by at least 10 nucleotides; and
a second plurality of complementary single-stranded helper nucleic acids hybridized with the first single-stranded nucleic acid, wherein the helper strands of the second plurality each include:
a first sequence hybridized to a first sequence of the first single-stranded nucleic acid; a second sequence hybridized to a second sequence of the first single-stranded nucleic acid; and a hairpin intermediate the first and second sequences of the helper strand; and
a plurality of nanocubes linked to helper nucleic acids by tethers.

15. A method of making a nanostructure, comprising:
selecting a first single-stranded nucleic acid having a known sequence;
determining a desired conformation for the first single-stranded nucleic acid;
selecting, based on the desired conformation, a first plurality of helper sequences configured to hybridize to selected sequences of the first single-stranded nucleic acid, thereby folding the first single-stranded nucleic acid into the desired conformation;
selecting a second plurality of helper sequences configured to hybridize to selected sequences of the first single-stranded nucleic acid, the second plurality of helper sequences each including at least one site capable of linking to a nanocube via a tether, and wherein the second plurality of helper sequences is selected to position linked nanocubes at desired locations in the nanostructure.

16. The method of claim 15, further comprising selecting a tether for each site capable of linking to a nanocube to position linked nanocubes at desired locations in the nanostructure.

17. The method of claim 16, wherein selecting the tether includes selecting a desired tether length.

18. The method of claim 15, further comprising forming the first plurality of helper strands, the second plurality of helper strands, and linking at least one nanocube to at least one of the second plurality of helper strands.

19. The method of claim 18, further comprising combining the first single-stranded nucleic acid, the first plurality of helper strands, and the second plurality of helper strands in a vessel; and allowing complementary sequences to hybridize, thereby forming the nanostructure.

20. The method of claim 19, wherein the nanostructure includes at least two nanocubes in proximity with one another.

21. The method of claim 20, further comprising causing the at least two nanocubes in proximity with one another to become bonded to one another.

22. The method of claim 21, wherein causing the at least two nanocubes in proximity with one another to become bonded to one another includes exposing the nanostructure to a trigger.

23. A method of making a nanostructure comprising combining in a vessel:
a first single-stranded nucleic acid having a known sequence;
a first plurality of complementary single-stranded helper nucleic acids capable of hybridizing with the first single-stranded nucleic acid, wherein the helper strands of the first plurality each include:
a first sequence capable of hybridizing to a first sequence of the first single-stranded nucleic acid; and a second sequence capable of hybridizing to a second sequence of the first single-stranded nucleic acid; wherein the first and second sequences of the first single-stranded nucleic acid are separated by at least 10 nucleotides; and
a second plurality of complementary single-stranded helper nucleic acids capable of hybridizing with the first single-stranded nucleic acid, wherein the helper strands of the second plurality each include:
a first sequence capable of hybridizing to a first sequence of the first single-stranded nucleic acid; a second sequence capable of hybridizing to a second sequence of the first single-stranded nucleic acid; and a nanocube linked to the helper strand by a tether; and
allowing the complementary sequences to hybridize, thereby forming the nanostructure.

24. The method of claim 23, wherein the nanostructure includes at least two nanocubes in proximity with one another.

25. The method of claim 24, further comprising causing the at least two nanocubes in proximity with one another to become bonded to one another.
